(19) United States
(12) Patent Application Publication
(10) Pub. No.: US 2016/0279206 A1
(43) Pub. Date: Sep. 29, 2016

(54) Methods and Compositions for the Treatment of Vaginal Diseases Employing Peroxide-Producing Enzymes and Peroxidases

(71) Applicant: Laclede, Inc., Rancho Dominguez, CA (US)

(72) Inventors: Michael Pellico, Rancho Dominguez, CA (US); Rajvinder Kaur Atwal, Rancho Dominguez, CA (US)

(21) Appl. No.: 15/151,315

(22) Filed: May 10, 2016

Related U.S. Application Data
(63) Continuation of application No. 12/445,239, filed on Dec. 18, 2009, now abandoned.
(60) Provisional application No. 60/828,933, filed on Oct. 10, 2006.

(51) Int. Cl. A61K 38/44; A61K 31/375; A61K 31/19; A61K 9/00; A61K 47/10

(52) U.S. Cl. CPC A61K 38/44; A61K 9/0034; A61K 47/10; A61K 31/19; A61K 31/375; C12Y 111/01007; C12Y 111/02002; C12Y 101/03004; A61K 38/443

(57) ABSTRACT

A therapeutic composition for vaginal administration based on the generation of a biocidal anion by an enzymatic reaction catalyzed by a peroxidase. The peroxide utilized by the peroxidase enzyme can be endogenous or can be generated by the action of an oxidase enzyme on a suitable substrate. Therapeutic compositions according to the present invention are useful for the treatment of vaginal diseases and conditions, including bacterial and fungal infections.

### CROSS-REFERENCES

This application is a Continuation Patent Application claiming the benefit of priority under 35 U.S.C. 120 from U.S. patent application Ser. No. 12/445,238 filed Apr. 10, 2009, which claims the benefit of priority under 35 U.S.C. 371 to International Patent Application No. PCT/US07/79840 filed Sep. 28, 2007, which claims the benefit of priority from U.S. Provisional Patent Application No. 60/828,933, filed Oct. 10, 2006, the entire contents of which are herein incorporated by reference.

### BACKGROUND OF THE INVENTION

This invention is directed to methods and compositions for the treatment of vaginal diseases employing peroxide-producing enzymes and peroxidases.

The healthy vagina has a number of natural protective factors against STD/HIV infection and related diseases. At puberty, with the onset of menstruation, the female genital tract undergoes changes due to the influence of the female sex hormone, estrogen. Soon after birth, the vagina becomes colonized with corynebacteria, staphylococci, nonpyogenic streptococci, Escherichia coil, and a lactic acid bacterium historically named “Doderlein's bacillus” (Lactobacillus acidophilus). During reproductive life, from puberty to menopause, the vaginal epithelium contains glycogen due to the actions of circulating estrogens. Doderlein's bacillus predominates, being able to metabolize the glycogen to lactic acid. The lactic acid and other products of metabolism inhibit colonization by all except Doderlein's bacillus and a select number of lactic acid bacteria. The resulting low pH of the vaginal epithelium prevents establishment of most bacteria as well as the potentially pathogenic yeast, Candida albicans. This is a striking example of the protective effect of the normal bacterial flora for their human host.

During puberty the previous thin and fragile vaginal mucosa grows plump and resilient, and becomes rich in glycogen (a carbohydrate that can be hydrolyzed into glucose by enzymatic reactions). Now, healthy organisms, the lactobacilli begin to thrive. Lactobacillus uses glycogen as an energy source, breaking it down into glucose and lactic acid. Under the influence of lactic acid, the vagina maintains a low pH of approximately 4.0. This acidic environment does two things: (1) it kills germs and (2) it causes squamous epithelial cells (mucous membrane) to cover over the exposed, fragile columnar cells of the cervical canal. Lactobacillus also produces hydrogen peroxide, which may kill some pathogens. Protective immune factors in the vagina include defensins, antibodies, nonspecific cytokines and inflammatory responses.

As previously mentioned, Lactobacillus species produce hydrogen peroxide especially Lactobacillus delbrueckii, Lactobacillus acidophilus, Lactobacillus crispatus, Lactobacillus johnsonii, and Lactobacillus gasseri. Hydrogen Peroxide reached concentrations from 0.05 to 1.0 mM, which under intensive aeration increased even up to 1.8 mM. Microorganisms related to vaginal pathologies show varied resistance to the action of pure hydrogen peroxide. Most potent inhibitory activity against bacteria and yeast was presented by Lactobacillus culture supernatant producing H₂O₂, followed by the nonproducing strain and pure H₂O₂. The antimicrobial activity of Lactobacilli is a summation of various inhibitory mechanisms in which H₂O₂ plays some but not a crucial role, in addition to other substances. A paper by Magdalena Strus, titled “The In Vitro Effect of Hydrogen Peroxide on Vaginal Microbial Communities” showed that hydrogen peroxide is important because of its role in the peroxidase antibacterial system.

The vagina is actually very good at taking care of itself if left to its own devices. Things can go wrong if anything happens to disrupt this normal, healthy balance. Common causes include antibiotics, overwashing, douching or the use of strong soaps, shower gels and vaginal deodorants. It is normal for woman to have a variable amount of vaginal discharge, which is usually white or clear. This may increase just before a period or during pregnancy. There are a number of common minor infections which can affect the vagina: this often causes a change from the normal type of discharge. It may become more strongly smelling, yellow or frothy. The discharge may occur with some external soreness, itching, burning or cracks in the skin. The commonest vaginal infections are: vaginal candidiasis, bacterial vaginosis and trichomoniasis.

A key part of the vagina's protection come from the peroxidase enzymes myeloperoxidase and lactoperoxidase. Another important antibacterial enzyme in the vagina is lysozyme. Although not an enzyme, the protein lactoferrin is also important for the vaginal defense system.

Myeloperoxidase is virucidal to immunodeficiency virus type 1 (HIV-1). Myeloperoxidase with the chloride ion present in medium did not require exogenous H₂O₂. The hydrogen peroxide comes from the HIV-1 infected cells. In the paper “Virucidal Effect of Myeloperoxidase on Human Immunodeficient HIV 1 Infected Cells,” the authors J Chochola and Y Yamaguchi, show that myeloperoxidase is virucidal to human immunodeficiency virus type 1 (HIV-1). The enzyme catalase partially inhibited the virucidal effect of myeloperoxidase.

The enzyme lactoperoxidase combines the bacteria produced H₂O₂ with the ions chloride, iodide, or with thiocyanate to produce a strong antibacterial and antifungal agent.

Lactoperoxidase is a key protective enzyme found in milk, the airway passages, saliva and the vagina. The enzyme converts hydrogen peroxide, a potentially harmful free radical, into an anti bacterial agent such as hypothiocyanite. Lactoperoxidase, along with other factors, helps control the vaginal flora and makes the environment suitable for the balanced growth of beneficial organisms. Lactoferrin is an iron binding protein that is found in the vagina, saliva, airway passages and in the intestines. Vaginal lactoferrin appears to be under hormonal control. Variations in vaginal lactoferrin concentration may result in alterations in susceptibility to bacterial pathogens such as Neisseria gonorrhoeae.

Unlike many traditional antibiotic agents, lactoferrin appears to exert its effect in several different ways. Primarily, lactoferrin binds to iron, making it unavailable for essential metabolic functions related to growth and reproduction/replication. In essence, one of the major mechanisms of action is to starve these organisms. Lactoferrin may also interfere with glucose uptake and metabolism. Lactoferrin also seems to interfere with the ability of non-living viruses to infect cells.

In the past vaginal infections have been treated with various antibacterial and antifungal agents such as nystatin, tetracycline, miconazole, clotrimazole, fluconazole, or itraconazole. The problem associated with most antibacterial agents is that they also kill beneficial organisms that are important to the health of the vagina. U.S. Patent Application Publication No. 2004/0126369 A1 by Payne at al. teaches about using peroxide—generating enzymes and substrates for the production of hydrogen peroxide. However some studies have shown that hydrogen peroxide can cause tissue damage and is not very effective as an antibacterial agent.

Introduction directly of lactobacilli into the vagina is not effective because very often the lactobacilli do not take hold. This happens because the environment for their growth is not ideal. If it were they would grow quite well without the need to introduce them.

Various peroxidases play an important role in protecting mammals from infections. The most important peroxidases are lactoperoxidase, myeloperoxidase, and eosinophil peroxidase. These various peroxidases have been found in saliva, milk, vaginal secretions, and recently in the lungs and sinuses. Peroxidase enzymes scavenge potentially toxic hydrogen peroxide and thus are also an important part of the body's defense against free radical damage.

In the mouth there is a need for defense against hydrogen peroxide because hydrogen peroxide is formed by bacteria colonizing the mucous membrane. In saliva, lactoperoxidase detoxifies hydrogen peroxide in the present of thiocyanate by converting it into hypothiocyanite (OSCN), molecular oxygen (O₂), and water. The hypothiocyanite ion then inhibits hydrogen-peroxide-producing bacteria. Lactoperoxidase thus forms a key part of the antibacterial defenses of saliva.

In milk the second most abundant protein is lactoperoxidase, In 1924 Hanssen suggested that the bacterial properties of milk against bacteria such as Salmonella species, including S. paratyphosa, are the results of its peroxidase activity. Since then numerous studies have confirmed its activity. From 1976 onwards Thomas and collaborators established ^{—}OSCN (hypothiocyanite ion) and its conjugate acid, HOSCN, as an oxidizing agent for bacterial sulfhydryls and proteins. In particular, the oxidation of the sulfhydryl groups of cysteine, an amino acid that is a constituent of proteins, into a disulfide can result in the denaturation and inactivation of the proteins. Many enzymes have a sulfhydryl group as part of their active site.

In the study “Isolation and Characterization of a Peroxidase from the Airway,” Salathe and Holderby showed that a peroxidase scavenges hydrogen peroxide from airways. Hydrogen peroxide is an important mediator of airway inflammation. They showed that this peroxidase was similar to lactoperoxidase but was different from other peroxidases including myeloperoxidase, eosinophil peroxidase, and glutathione peroxidases. As in the oral cavity and vagina, the peroxidase controls free radicals and catalyzes the function of biocidal compounds. This is especially important during times of infection. For example, the bacterium Streptococcus pneumoniae produces large amounts of hydrogen peroxide which inflames lung tissue. The authors designated the peroxidase activity found in tracheal secretions airway peroxidase (APO). This peroxidase, like lactoperoxidase in saliva, is likely to be biocidal against bacteria, fungi, and viruses and to act as a scavenger of hydrogen peroxide during airway inflammation. In a study published in 2000 entitled “The Lactoperoxidase System Functions in Bacterial Clearance of Airways” by Gersen, Sabater, and Scuri, the airway peroxidase was shown to be identical to milk lactoperoxidase. Their data also showed that the lactoperoxidase system is a major contributor to airway defense systems. As described earlier, the lactoperoxidase system is a significant free radical scavenger. Studies have shown that S. pneumoniae infections are associated with significant damage to the alveolar epithelium.

As in other parts of the body, the lactoperoxidase system, along with other peroxidase, lysozyme, and lactoferrin, usually works quite well in purging the body of harmful organisms. However, in times of severe infections, this protective system can be overwhelmed. Besides infections, another potential cause of high levels of hydrogen peroxide is found in patients suffering from acute respiratory failure or from ARDS (acute respiratory distress syndrome). Patients with acute respiratory failure or ARDS exhibit higher concentrations of hydrogen peroxide than control patients.

Several patents and patent applications describe the use of an enzymatic system to produce an antibacterial or biocidal effect. U.S. Pat. No. 4,370,199 to Orndorff (1983) discloses a method of killing and inhibiting the growth of microorganisms in industrial process streams by the addition of an enzymatically catalyzed biocide system which utilized a plant dehydrogenase enzyme such as horseradish peroxidase in the presence of an oxidant such as hydrogen peroxide to oxidize a halide salt such as potassium iodide or sodium chloride to produce an oxidation product that is toxic to microorganisms.

U.S. Pat. No. 4,150,113 to Hoogendoorn et al. (1979) and U.S. Pat. No. 4,178,362 to Hoogendoorn et al. (1979) disclose, respectively, an enzymatic toothpaste and an enzymatic chewable dentifrice containing glucose oxidase which acts on glucose present in saliva and tooth plaque to produce hydrogen peroxide. The patentees note that oral bacteria, through enzyme systems having sulfhydryl groups, effect glycolysis of food products containing sugars and point out that lactoperoxidase, which is present in saliva, provides the means for transferring oxygen from hydrogen peroxide to oral bacteria resulting in the oxidation of the sulfhydryl-group-containing enzymes into inactive enzymes in which the sulfhydryl groups have been oxidized into disulfide groups. It is further disclosed that the dentifrice can be formulated with potassium thiocyanate.

U.S. Pat. No. 4,269,822 to Pellico et al. (1981) discloses an antiseptic dentifrice containing an oxidizable amino acid substrate and an oxidoreductase enzyme specific to the substrate for producing hydrogen peroxide and ammonia upon oral application of the dentifrice, with pre-application stability being maintained by limiting the quantity of any water present in the dentifrice.

U.S. Pat. No. 4,537,764 to Pellico et al. (1985) discloses an enzymatic dentifrice containing β-D-glucose and glucose oxidase for producing hydrogen peroxide upon oral application of the dentifrice, with pre-application stability being maintained by limiting any water in the dentifrice to not more than about 10% by weight based on the weight of the dentifrice.

U.S. Pat. No. 4,576,817 to Montgomery et al. (1986) discloses enzymatic bandages and pads, for body contact applications, containing, for example, glucose oxidase which catalyzes a reaction between β-D-glucose, water, and oxygen in serum to produce hydrogen peroxide. The bandages and pads can further contain a peroxidase and an oxidizable salt such as thiocyanate, chloride, or iodide salts of sodium or potassium which, in the presence of hydrogen peroxide and peroxidase, are oxidized to hypothiocyanite, hypochlorite, and hypoiodite, respectively, and that function as bacterial inhibitors.

U.S. Pat. No. 4,564,519 to Pellico et al. (1986) discloses a di-enzymatic chewable dentifrice which, contains, for example, glucose and glucose oxidase for producing hydrogen peroxide upon chewing the dentifrice and further contains a thiocyanate salt and lactoperoxidase for reacting with the hydrogen peroxide to produce a hypothlocyanite bacterial inhibitor, with pre-application stability being maintained by limiting any unbound water in the chewable dentifrice to an amount of not more than about 1.0 weight percent, and by limiting the total water, bound and unbound, to not more than about 10 weight percent by weight. U.S. Pat. No. 4,578,365 to Pellico et al. (1986) discloses a di-enzymatic dentifrice which contains, for example, glucose and glucose oxidase for producing hydrogen peroxide upon oral application of the dentifrice and further contains a thiocyanate salt and lactoperoxidase for reading with the hydrogen peroxide to produce a hypothlocyanite, with pre-application stability being maintained by limiting any water in the dentifrice to not more than about 10 weight percent based on the weight of the dentifrice.

U.S. Pat. No. 4,617,190 to Montgomery (1986) discloses an enzymatic powdered milk that contains, for example, glucose, glucose oxidase, a peroxidase, and potassium iodide for producing hypoiodite, an anionic bacterial inhibitor in the reconstituted milk.

U.S. Pat. No. 5,336,494 to Pellico (1994) discloses an orally chewable, enzymatically coated pet product, which contains, for example, β-D-glucose and glucose oxidase for producing hydrogen peroxide upon oral chewing of the product, and can further contain a peroxidase and an alkali metal salt of an oxygen accepting anion such as potassium iodide for reaction with hydrogen peroxide to produce hypoiodite, an anionic bacterial inhibitor.

U.S. Pat. No. 5,453,284 to Pellico (1995) discloses an aqueous enzymatic dentifrice having a water content in excess of 10 weight percent and which contains, for example, β-D-glucose and glucose oxidase for producing hydrogen peroxide upon oral application of the dentifrice and can further contain a peroxidase and an oxidizable alkali metal sett such as the thiocyanate, chloride, or iodide salt of sodium or potassium for reacting with hydrogen peroxide to produce an anionic bacterial inhibitor. Pre-application stability is maintained by the addition of a water-soluble thickener in a quantity such that the dentifrice has a viscosity from about 800 to about 75,000 centipoises.

Accordingly, there is a need for improved methods and compositions to treat vaginal diseases and conditions, particularly vaginal diseases and conditions that are bacterial or fungal in origin. There is a further need for improved methods and compositions that are safe and can be used together with other antibacterial or antifungal components. In particular, there is a need for improved methods and conditions that will encourage the growth of lactobacilli in the vagina, which then exert natural bidogical control over the growth of bacterial and fungal pathogens.

### SUMMARY OF THE INVENTION

This invention entails the introduction into the vagina the complete peroxidase system. This system comprises a peroxidase such as lactoperoxidase or myeloperoxidase and a substrate such as potassium thiocyanate. This system requires hydrogen peroxide which is present in the vagina. If not enough hydrogen peroxide is present in the vagina, then this invention has, as part of it, the addition of an oxidoreductase enzyme and its specific substrate. This enzyme system in this invention will provide an ideal growth environment for lactobacilli. The lactobacilli will then inhibit the growth of pathogenic bacteria and also prevent the overgrowth of yeast.

The composition can further comprise an effective amount of an inhibitor that is specific for catalase. Typically, the inhibitor that is specific for catalase is a salt of ascorbic acid. Typically, the salt of ascorbic acid is selected from the group consisting of sodium ascorbate, potassium ascorbate, calcium ascorbate, ascorbyl palmitate, and mixtures thereof. The composition can further comprise an iron salt; typically, the iron salt is selected from the group consisting of ferrous sulfate, ferrous chloride, and ferrous iodide.

The composition can further comprise a quantity of an aminohexose effective in increasing the yield or accumulation of biocide formed. Typically, the aminohexose is an aminoglucose. Typically, the aminoglucose is selected from glucosamine, N-acetylglucosamine, and mixtures thereof.

In the composition, the media can be each independently selected from the group consisting of water, glycerol, sorbitol, propylene glycol, and mixtures thereof, with the proviso that at least one of the media includes a substantial proportion of water.

The composition can further comprise a buffering agent. Typically, the buffering agent is selected from the group consisting of sodium stearate, potassium stearate, and calcium stearate.

The composition can further comprise any or all of lysozyme, lactoferrin, or a steroid. Typically, the steroid is selected from the group consisting of hydrocortisone, beclomethasone, budenoside, ciclesonide, flunisolide, fluticasone, methylprednisolone, prednisolone, prednisone, and triamcinolone, and the salts, solvates, analogues, congeners, bioisosteres, hydrolysis products, metabolites, precursors, and prodrugs thereof. Preferably, the steroid is hydrocortisone.

Another embodiment of a therapeutic composition according to the present invention is a composition comprising:

(1) a peroxidase enzyme that catalyzes a reaction between hydrogen peroxide and a salt that acts as an oxygen acceptor and is capable of reacting with hydrogen peroxide to form a biocide, the peroxidase enzyme being present in a sufficient quantity such that the biocide is produced in a therapeutically effective concentration;
(2) a salt that acts as an oxygen acceptor and is capable of reacting with hydrogen peroxide to form a biocide in a quantity sufficient to form a therapeutically effective concentration of the biocide; and
(3) an aqueous medium in which the peroxidase enzyme and the salt that acts as an oxygen acceptor are stable.

### DETAILED DESCRIPTION OF THE INVENTION

One embodiment of the present invention is a therapeutic composition for vaginal administration comprising:
(1) a first component comprising:
   (a) one of:
      (i) an oxidoreductase enzyme that produces hydrogen peroxide by catalyzing the oxidation of a substrate for which the oxidoreductase enzyme is specific, the first component comprising a sufficient quantity of the oxidoreductase enzyme that a quantity of hydrogen peroxide sufficient to react with a peroxidase is produced; and
      (ii) a substrate that is oxidizable in a reaction catalyzed by the oxidoreductase enzyme in a sufficient quantity that a quantity of hydrogen peroxide sufficient to react with a peroxidase is produced;
   (b) a peroxidase enzyme that catalyzes a reaction between hydrogen peroxide and a salt that acts as an oxygen acceptor and is capable of reacting with hydrogen peroxide to form a biocide, the peroxidase enzyme being present in a sufficient quantity such that the biocide is produced in a therapeutically effective concentration; and
   (c) an aqueous or nonaqueous medium in which the enzymes and the oxidizable substrate, if present, are stable;
(2) a second component comprising:
   (a) the other of the oxidoreductase enzyme and the substrate that is oxidizable in a reaction catalyzed by the oxidoreductase enzyme that is not present in (1);
   (b) a salt that acts as an oxygen acceptor and is capable of reacting with hydrogen peroxide to form a biocide in a quantity sufficient to form a therapeutically effective concentration of the biocide; and
   (c) an aqueous or nonaqueous medium in which the other of the oxidoreductase enzyme and the oxidizable substrate and the salt that acts as an oxygen acceptor are stable, with the proviso that one of the media of the first component and the second component is aqueous, such that the composition is suitable for vaginal administration.

This embodiment is particularly suitable for the treatment of diseases and conditions such as those caused by fungus in which there is no additional endogenous hydrogen peroxide or only a minimal quantity of endogenous hydrogen peroxide produced by the disease process. In this embodiment, therefore, an oxidizable substrate and an oxidoreductase enzyme specific for the substrate is added in order to ensure an adequate amount of hydrogen peroxide to create an effective quantity of biocide.

In one alternative of the composition as described above, the first component includes the oxidoreductase enzyme. In another alternative of the composition as described above, the first component includes the oxidizable substrate.

Typically, the composition comprises from about 0.6 to about 500 International Units of the oxidoreductase enzyme. Typically, the composition comprises from about 0.015 to about 0.6 millimole of the oxidizable substrate. Typically, the composition comprises from about 0.05 to about 30 International Units of the peroxidase enzyme. Typically, the composition comprises from about 0.0001 to about 0.01 millimole of the salt that acts as an oxygen acceptor.

In one alternative, the media of the first and second component are both aqueous media. In another alternative, the medium of the first component can be a nonaqueous medium such as glycerol. As used herein, the term “aqueous” does not exclude nonaqueous ingredients such as glycerol or sorbitol, as long as a significant proportion of water is present in the medium.

More than one peroxidase enzyme can be included. For example, both lactoperoxidase and horseradish peroxidase can be used.

As used herein, the term International Unit (IU) is defined as the quantity of enzyme that catalyzes the conversion of one micromole of substrate per minute under defined standard assay conditions for that enzyme.

The oxidoreductase enzyme is typically selected from the group consisting of glucose oxidase, galactose oxidase, urate oxidase, choline oxidase, D-amino acid oxidase, D-glutamate oxidase, glycine oxidase, glycolic oxidase, sorbose oxidase, alcohol oxidase, and amine oxidase. Other enzymes can alternatively be used, such as nitroethane oxidase, D-aspartate oxidase, L-aminoacid oxidase, pyridoxamine phosphate oxidase, ethanolamine oxidase, pyruvateoxidase, oxalate oxidase, hexose oxidase, cholesterol oxidase, aryl alcohol-,oxidase, pyridoxine 4-oxidase, dehydroorotate oxidase, lathosterol oxidase, sarcosine oxidase, N-methylaminoacid oxidase, N⁶-methyllysine oxidase, 6-hydroxy-L-nicotine oxidase, 6-hydroxy-D-nicotine oxidase, 3-hydroxyanthranilate oxidase, aldehyde oxidase, and xanthine oxidase, as described in U.S. Pat. No. 4,340,448 to Schiller et al., incorporated herein by this reference.

For these enzymes, glucose oxidase catalyzes the reaction of β-D-glucose, water, and oxygen to produce hydrogen peroxide and gluconic acid. Galactose oxidase catalyzes the reaction of D-galactose and oxygen to produce hydrogen peroxide and D-galacto-hexodialdose. Urate oxidase catalyzes the reaction of uric add, water, and oxygen to produce hydrogen peroxide, allantoin, and carbon dioxide. Choline oxidase catalyzes the reaction of choline and oxygen to produce hydrogen peroxide and betaine aldehyde. D-amino acid oxidase catalyzes the reaction of D-amino acids such as D-proline, D-methionine, D-isoleucine, D-alanine, D-valine, or D-phenylalanine with water and oxygen to produce hydrogen peroxide, ammonia, and the α-keto acid corresponding to the D-amino acid being oxidized. D-glutamate oxidase catalyzes the reaction of D-glutamic acid, water, and oxygen to produce hydrogen peroxide, ammonia, and 2-ketoglutarate. Glycine oxidase catalyzes the reaction of glycine, water, and oxygen to produce hydrogen peroxide, ammonia, and glyoxylic acid. Glycolic acid oxidase (also known as 2-hydroxyacid oxidase) catalyzes the reaction of glycolic acid and oxygen to produce 2-ketoacetic acid and hydrogen peroxide. L-sorbose oxidase catalyzes the reaction of L-sorbose and oxygen to produce 5-dehydro-D-fructose and hydrogen peroxide. Alcohol oxidase catalyzes the reaction of a lower primary alcohol or an unsaturated alcohol and oxygen to produce the corresponding aldehyde and hydrogen peroxide. Amine oxidase catalyzes the reaction of an amine, typically a primary amine, but also, in some cases, a secondary or tertiary amine, water, and oxygen to produce the corresponding aldehyde, ammonia, and hydrogen peroxide. In an illustrative reaction, glucose oxidase catalyzes the reaction of β-D-glucose, water, and oxygen during application to the outer ear to produce hydrogen peroxide and gluconic acid.

The peroxidase enzyme is typically one of lactoperoxidase, horseradish peroxidase, myeloperoxidase, eosinophil peroxidase, and glutathione peroxidase.

The salt that acts as an oxygen acceptor and is capable of reacting with hydrogen peroxide to form a biocide is typically an alkali metal salt of an anion such as thiocyanate, iodate, or chlorate. The alkali metal salt is typically a sodium or potassium salt, although other alkali metal salts such as lithium or cesium can alternatively be used.

The properties of a number of preferred oxidases suitable for use in compositions according to the present invention are known. For example, glucose oxidase from Aspergillus niger has been determined to have a molecular weight of 150,000 (Pazur et al. (1965)). The enzyme is a glycoprotein containing two molecules of the redox coenzyme flavin adenine dinucleotide (FAD). The amino acid composition has been determined. The isoelectric point of the enzyme is 4.2. The optimum pH of the enzyme is 5.5 with a broad pH range of from 4 to 7. Inhibitors of the enzyme include monovalent silver ions and divalent mercury and copper ions.

Galactose oxidase from Dactylium dendroides has a molecular weight of 42,000. It is a metalloenzyme containing one gram-atom of copper per mole. The amino acid composition has been determined. The optimum pH of the enzyme is 7.

Urate oxidase (uricase) from hog liver or beef liver has a molecular weight of 100,000. It is a metalloenzyme containing one gram-atom of copper per mole. The isoelectric point of the enzyme is 6.3. The optimum pH of the enzyme is 9.

D-amino acid oxidase from hog kidney has a molecular weight of 90,000. The enzyme is a glycoprotein containing two molecules of flavin adenine dinucleotide. The optimum pH of the enzyme is 9.1. Certain heavy metals are inhibitors of the enzyme.

The oxidizable substrate is typically present in the therapeutic composition at a concentration of from about 0.015 millimoles per milliliter of liquid to about 0.6 millimoles per gram of composition. Preferably, the oxidizable substrate is present in the therapeutic composition at a concentration of from about 0.025 millimoles per gram of composition to about 0.1 millimole per gram of composition. The salt that acts as an oxygen acceptor is typically present in the therapeutic composition at a concentration of from about 0.0001 millimole to about 0.01 millimole per gram of composition. The salt that acts as an oxygen acceptor is preferably present in the therapeutic composition at a concentration of from about 0.001 millimole to about 0.006 millimole per gram of composition.

Typically, the oxidoreductase enzyme is present in the therapeutic composition in a concentration of from about 0.5 IU to about 500 IU per gram of composition. Preferably, the oxidoreductase enzyme is present in the therapeutic composition in a concentration of from about 10 IU to about 40 IU per gram of composition. Oxidoreductase enzymes are supplied in dry or liquid form with the label specifying the concentration in international Units on a per gram or per milliliter basis, as appropriate.

As indicated above, the therapeutic composition according to the present invention is also provided with a second enzyme. The second enzyme is a peroxidase. A suitable peroxidase is lactoperoxidase. Lactoperoxidase is a glycoprotein which, in one commercial embodiment, is a lyophilized powder derived from milk. This commercial peroxidase has an activity of 80 IU/mg and a projected molecular weight of 93,000 for L-tyrosine iodination. The physicochemical properties reported for lactoperoxidase include a molecular weight of 78,000, a partial specific volume, reflective of the amino acid composition, of 0.74, and the presence of 1.0 mole of heme per mole of lactoperoxidase. As indicated above, other peroxidases, including, but not limited to, horseradish peroxidase, myeloperoxidase, eosinophil peroxidase, and glutathione peroxidase, can alternatively be used.

The peroxidase is typically present in the therapeutic composition in a concentration of from about 0.05 IU to about 30 IU per gram of composition; preferably, the peroxidase is present in the therapeutic composition in a concentration of from about 0.1 IU to about 1.0 IU per gram of composition.

The operable integrity of the enzymatic system can be affected by the presence of catalase, which is present in commercial glucose oxidase as well as in mucous membrane tissue. Catalase, which is extraneous to the enzymatic system of this invention, competes with peroxidase for hydrogen peroxide. In order to reduce the loss of hydrogen peroxide through the presence of catalase, an effective amount of an enzymatic inhibitor that is specific for catalase can be advantageously incorporated into a therapeutic composition according to the present invention. Suitable enzymatic inhibitors specific for catalase include, but are not limited to ascorbic salts such as sodium ascorbate, potassium ascorbate, calcium ascorbate, ascorbyl palmitate, or mixtures thereof, and can be included in a therapeutic composition according to the invention. An effective concentration of ascorbic salt in compositions according to the present invention is from about 1×10⁻⁶ to about 1×10⁻⁴ millimole per gram of therapeutic composition. Iron salts such as ferrous sulfate, ferrous chloride, or ferrous iodide can also be incorporated into a therapeutic composition according to the present invention as a potentiator for the ascorbic salt in its role as catalase inhibitor. A particularly preferred iron salt is ferrous sulfate.

Therapeutic compositions according to the present invention can also advantageously be formulated with an aminohexose in order to increase the yield or accumulation of oxidized anionic biocidal agent, the quantity of the aminohexose being effective to increase the yield or accumulation of oxidized anionic biocidal agent. Typically, the aminohexose is an aminoglucose, but other aminohexoses such as aminogalactose can alternatively be used. Typically, the aminoglucose is selected from the group consisting of glucosamine, N-acetylglucosamine, and mixtures thereof. The aminoglucose is typically present in the therapeutic composition in a concentration of from about 0.0001 millimole to about 0.002 millimole per gram of composition. Preferably, the aminoglucose is present in the therapeutic composition in a concentration of from about 0.0003 millimole to about 0.001 millimole per gram of composition.

The media described above typically are each independently selected from the group consisting of water, glycerol, sorbitol, propylene glycol, and mixtures thereof, with the proviso that at least one of the media includes a substantial proportion of water. As used herein, the term “substantial proportion of water” is defined as a sufficient quantity of water when the two components are mixed so that ions can be efficiently solvated and that enzymatic reactions that require the participation of ionic species can proceed efficiently. In addition, nonaqueous media can include solvents with substantially equivalent properties that are non-denaturing with respect to the enzymes and serve as suitable media for catalysis of the reactions catalyzed by the enzymes. The media are typically present in the composition in a total concentration from about 80 weight percent to about 96 weight percent. Preferably, the media are present in the composition in a total concentration from about 90 weight percent to about 96 weight percent. The media and the concentration thereof are selected such as to provide the composition with appropriate pressure responsive application characteristics. Typically, the media act as a lubricant. Other ingredients can be included in the media.

In some alternatives, the products of the activated enzyme system of the therapeutic composition include a weak organic acid, such as gluconic acid. In this case, it is advantageous to formulate the composition with a buffering agent in order to neutralize the organic acid. Suitable buffering agents include, but are not limited to, salts of stearic acid such as sodium stearate, potassium stearate, or calcium stearate. A particularly preferred salt of stearic acid is sodium stearate. These salts can be present in the composition in a concentration of up to about 6.0 weight percent. Typically, the salt is present in the composition in an amount of from about 2.0 weight percent to about 6.0 weight percent. Citric add can also be used as a buffering agent.

The composition can further include a salt of sorbic acid such as sodium sorbate or potassium sorbate. A preferred salt of sorbic acid is potassium sorbate.

Adjunct therapeutic agents such as the enzyme lysozyme, the protein lactoferrin, and an anti-inflammatory medication such as a steroid, including, but not limited to, hydrocortisone, beclomethasone, budenoside, ciclesonide, flunisolide, fluticasone, methylprednisolone, prednisolone, prednisone, and triamcinolone, as well as the salts, solvates, analogues, congeners, bioisosteres, hydrolysis products, metabolites, precursors, and prodrugs thereof, can be added to the enzymatic formulations of this invention. A particularly preferred steroid is hydrocortisone.

Other ingredients generally known in the pharmaceutical art can be incorporated into therapeutic compositions according to the present invention, including colorants, chelating agents, preservatives, and stabilizers, with the proviso that these additional ingredients do not inhibit the oxidation-reduction reactions on which the activity of the compositions according to the present invention depend.

In another embodiment of the invention, the oxidoreductase enzyme and the substrate that is oxidizable are omitted. In this embodiment, the composition includes the peroxidase enzyme and the salt that acts as an oxygen acceptor, and the composition acts by degrading endogenous hydrogen peroxide, such as occurs in vaginal tissues and elsewhere in the body.

In general, this embodiment of the composition comprises:
(1) a peroxidase enzyme that catalyzes a reaction between hydrogen peroxide and a salt that acts as an oxygen acceptor and is capable of reacting with hydrogen peroxide to form a biocide, the peroxidase enzyme being present in a sufficient quantity such that the biocide is produced in a therapeutically effective concentration;
(2) a salt that acts as an oxygen acceptor and is capable of reacting with hydrogen peroxide to form a biocide in a quantity sufficient to form a therapeutically effective concentration of the biocide; and
(3) an aqueous medium in which the peroxidase enzyme and the salt that acts as an oxygen acceptor are stable.

The peroxidase enzyme and the salt that acts as an oxygen acceptor are as described above.

In this alternative, typically, the composition comprises from about 0.05 to about 30 International Units of the peroxidase enzyme. Typically, the composition comprises from about 0.0001 to about 0.01 millimole of the salt that acts as an oxygen acceptor.

As described above, this embodiment of the composition can further comprise an effective amount of an inhibitor that is effective for catalase. This embodiment of the composition can further comprise an iron salt, as described above. This embodiment of the composition can also further comprise a quantity of an aminohexose effective in increasing the yield or accumulation of biocide formed, as described above. This embodiment of the composition can also further comprise a buffering agent, as described above. In addition, this embodiment of the composition can further comprise any or all of lysozyme, lactoferrin, or a steroid, as described above.

In one alternative, a therapeutic composition according to the present invention that comprises a hydro-activated and/or oxygen-activated aqueous enzymatic, antimicrobial lubricant is stabilized against enzymatic activation prior to vaginal application by incorporating a thickener into the formulation so as to provide the formulation with an enzyme immobilizing viscosity which inhibits enzymatic action during processing and in packing. Non-aqueous enzymatic lubricants do not need a thickener as stabilizer. An illustrative thickened enzymatic lubricant with this enhancement contains glucose oxidase, glucose, lactoperoxidase, myeloperoxidase and potassium thiocyanate together with carboxymethyl cellulose and xanthan gum in an amount to provide the lubricant with a viscosity of at least about 700 cps. Preferably, the viscosity is from about 700 cps to about 100,000 cps for liquids and thin gels containing water. Other thickeners are known in the art and can be alternatively used. These thickeners include hydroxymethyl cellulose, methyl cellulose, polyvinylpyrrolidone (PVP), PVM, PVM/MA copolymers, and mixtures thereof.

Typically, when the formulation is an aqueous formulation, the water content is from about 7% to about 60% of the therapeutic composition. However, as described below, the formulation can be a non-aqueous formulation with substantially no water content.

The physical form of a formulation according to the present invention can be, for example, a solution, a gel, a cream, or a solid such as a suppository. If the solution is a gel, the viscosity of the gel can be chosen to provide efficient application by the user according to general principles of gel formulation for pharmaceutical compositions. The particular gel former or gel formers used in a particular formulation and their concentrations can be determined by one of ordinary skill in the art. Typically, formulations according to the present invention act as a lubricant in the vagina.

Formulations according to the present invention can include additional components, such as, but not limited to, a gel forming component, a lipophilic component, a wax, a skin soothing component, an emulsifier component, a bulk adding component, a gum component, or other components such as are generally used in pharmaceutical compositions intended for vaginal application, such as stabilizers, buffers, a colorant, a fragrance, or a preservative. In particular, formulations according to the present invention can include one or more of the following components: (1) caprylic/capric triglycerides; (2) glycerol; (3) dipropylene glycol; (4) tripropylene glycol; (5) xanthan gum; (6) PEG-20 almond glyceride; (7) an isopropyl ester of a long chain fatty acid selected from the group consisting of isopropyl myristate, isopropyl laurate, and isopropyl stearate, preferably isopropyl myristate; (8) aloe Vera; (9) sodium polyacrylate/polyacrylic acid; (10) beeswax; (11) PEG-40 stearate; (12) polyethylene glycol; and (13) Polawax.

Therapeutic compositions according to the present invention can be formulated by techniques known in the art, including techniques that are conventional in the cosmetic art and in the art of over-the-counter and prescription drug formulation for blending lipid-soluble components and water-soluble components for the preparation of liquids, gels, creams, or suppositories. These mixing techniques include both manual and mechanical mixing, and include homogenization mixing and sweep mixing. The mixing techniques to be used can be chosen by one of ordinary skill in the art based on variables such as the viscosity of the components to be mixed and the volume of those components, as well as the relative proportion of lipid-soluble and water-soluble ingredients, the proportion of water, and the final physical form of the desired formulation.

Particular embodiments of therapeutic compositions according to the present invention, include, but are not limited to the following:
Formulation 1 is an aqueous enzymatic lubricant containing about 50% water together with an enzyme system including D-glucose, glucose oxidase, lactoperoxidase, and potassium thiocyanate.
   Typically, Formulation 1 comprises:
(1) from about 40% to about 60% of water;
(2) from about 19.2% to about 28.8% of 99% glycerol;
(3) from about 8% to about 12% of carboxymethyl cellulose;
(4) from about 11.2% to about 16.8% of caprylic/capric triglycerides;
(5) from about 0.012% to about 0.018% of lactoperoxidase;
(6) from about 0.012% to about 0.018% of myeloperoxidase;
(7) from about 0.024% to about 0.036% of glucose oxidase; and
(8) from about 0.008% to about 0.012% of sodium phosphate.
   Preferably, Formulation 1 comprises:
(1) about 50% water;
(2) about 24% of 99% glycerol;
(3) about 10% of carboxymethyl cellulose;
(4) about 14% of caprylic/capric triglycerides;
(5) about 0.015% of lactoperoxidase;
(6) about 0.015% of myeloperoxidase;
(7) about 0.030% of glucose oxidase; and
(8) about 0.010% of sodium phosphate.
   Formulation 2 is an aqueous enzymatic lubricant comprising 30% of water together with an enzymatic system.
   Typically, Formulation 2 comprises:
(1) from about 24% to about 36% of water;
(2) from about 20% to about 30% of glycerol;
(3) from about 24% to about 30% of tripropylene glycol;
(4) from about 8% to about 12% of caprylic/capric triglycerides;
(5) from about 2.4% to about 3.6% of carboxymethyl cellulose;
(6) from about 2.4% to about 3.6% of xanthan gum;
(7) from about 0.028% to about 0.042% of lactoperoxidase; and
(8) from about 0.024% to about 0.036% of glucose oxidase.
   Preferably, Formulation 2 comprises:
(1) about 30% of water;
(2) about 25% of glycerol;
(3) about 30% of tripropylene glycol;
(4) about 10% of caprylic/capric triglycerides;
(5) about 3.0% of carboxymethyl cellulose
(6) about 3.0% of xanthan gum;
(7) about 0.035% of lactoperoxidase; and
(8) about 0.030% of glucose oxidase.
   Formulation 3 is an aqueous enzymatic lubricant comprising 7% of water together with an enzyme system.
   Typically, Formulation 3 comprises:
(1) from about 5.6% to about 8.4% of water;
(2) from about 48% to about 72% of 99% glycerol;
(3) from about 5.6% to about 8.4% of tripropylene glycol;
(4) from about 8% to about 12% of caprylic/capric triglycerides;
(5) from about 4.8% to about 7.2% of carboxymethyl cellulose;
(6) from about 8% to about 12% of PEG-20 almond glyceride;
(7) from about 0.028% to about 0.042% of lactoperoxidase; and
(8) from about 0.024% to about 0.036% of glucose oxidase.
   Preferably, Formulation 3 comprises:
(1) about 7.0% of water;
(2) about 60% of 99% glycerol;
(3) about 7.0% of tripropylene glycol;
(4) about 10% of caprylic/capric triglycerides;
(5) about 6.0% of carboxymethyl cellulose;
(6) about 10% of PEG-20 almond glyceride;
(7) about 0.035% of lactoperoxidase; and
(8) about 0.030% of glucose oxidase.

Formulation 4 is an aqueous enzymatic lubricant in cream form.

Typically, Formulation 4 comprises:
(1) from about 10.4% to about 15.6% of water;
(2) from about 20% to about 80% of 99% glycerol;
(3) from about 16% to about 24% of glyceryl polymethacrylate;
(4) from about 22.08% to about 33.12% of sodium polyacrylate/polyaclylic acid;
(5) from about 4.0% to about 6.0% of propylene glycol;
(6) from about 4.0% to about 6.0% of caprylic/capric triglycerides;
(7) from about 3.2% to about 4.8% of xanthan gum;
(8) from about 0.24% to about 0.36% of isopropyl myristate;
(9) from about 0.024% to about 0.036% of aloe vera;
(10) from about 0.012% to about 0.018% of lactoperoxidase;
(11) from about 0.012% to about 0.018% of horseradish peroxidase;
(12) from about 0.024% to about 0.036% of glucose oxidase; and
(13) from about 0.004% to about 0.006% of potassium iodide.

In Formulation 4, in place of isopropyl myristate, another isopropyl ester of a long-chain fatty acid can be used, including, but not limited to, isopropyl laurate and isopropyl stearate.

Preferably, Formulation 4 comprises:
(1) about 13% of water;
(2) about 25% of 99% glycerol;
(3) about 20% of glyceryl polymethacrylate;
(4) about 27.6% of sodium polyacrylate/polyacrylic acid;
(5) about 5.0% of propylene glycol;
(8) about 5.0% of caprylic/capric triglycerides;
(7) about 4.0% of xanthan gum;
(8) about 0.30% of isopropyl myristate;
(9) about 0.030% of aloe vera;
(10) about 0.015% of lactoperoxidase;
(11) about 0.015% of horseradish peroxidase;
(12) about 0.030% of glucose oxidase; and
(13) about 0.005% of potassium iodide.

Formulation 5 is a non-aqueous enzymatic lubricant in gel form. The viscosity of this non-aqueous lubricant is about 80,000 cps.

Typically, Formulation 5 comprises:
(1) from about 51.2% to about 76.8% of 99% glycerol;
(2) from about 4% to about 6% of tripropylene glycol;
(3) from about 11.2% to about 16.8% of caprylic/capric triglycerides;
(4) from about 4.8% to about 7.2% of carboxymethyl cellulose;
(5) from about 3.2% to about 4.8% of xanthan gum;
(6) from about 8.0% to about 12.0% of PEG-20 almond glyceride;
(7) from about 0.012% to about 0.018% of lactoperoxidase;
(8) from about 0.012% to about 0.018% of horseradish peroxidase; and
(9) from about 0.024% to about 0.036% of glucose oxidase.
   Preferably, Formulation 5 comprises:
(1) about 64% of 99% glycerol;
(2) about 5% of tripropylene glycol;
(3) about 14% of caprylic/capric triglycerides;
(4) about 6.0% of carboxymethyl cellulose;
(5) about 4.0% of xanthan gum;
(6) about 10% of PEG-20 almond glyceride;
(7) about 0.015% of lactoperoxidase;
(8) about 0.015% of horseradish peroxidase; and
(9) about 0.030% of glucose oxidase.

Formulation 6 is a non-aqueous enzymatic lubricant in cream form.

Typically, Formulation 6 comprises;
(1) from about 20.8% to about 31.2% of 99% glycerol;
(2) from about 8.0% to about 12.0% of tripropylene glycol;
(3) from about 8.0% to about 12.0% of caprylic/capric triglycerides;
(4) from about 3.2% to about 4.8% of xanthan gum;
(5) from about 4.0% to about 6.0% of PEG-20 almond glyceride;
(6) from about 4.0% to about 6.0% of polyethylene glycol;
(7) from about 32% to about 48% of sodium polyacrylate/polyacrylic acid;
(8) from about 0.024% to about 0.036% of lactoperoxidase;
(9) from about 0.0040% to about 0.0060% of myeloperoxidase;
(10) from about 0.012% to about 0.018% of lactoferrin; and
(11) from about 0.012% to about 0.018% of lysozyme.
   Preferably, Formulation 6 comprises:
(1) about 26% of 99% glycerol;
(2) about 10.0% of tripropylene glycol;
(3) about 10.0% of caprylic/capric triglycerides;
(4) about 4.0% of xanthan gum;
(5) about 5.0% of PEG-20 almond glyceride;
(6) about 5.0% of polyethylene glycol;
(7) about 40% of sodium polyacrylate/polyacrylic acid;
(8) about 0.030% of lactoperoxidase;
(9) about 0.0050% of myeloperoxidase;
(10) about 0.015% of lactoferrin; and
(11) about 0.015% of lysozyme.

Formulation 7 is a non-aqueous enzymatic lubricant in gel form.

Typically, Formulation 7 comprises:
(1) from about 16% to about 24% of 99% glycerol;
(2) from about 8.0% to about 12.0% of tripropylene glycol;
(3) from about 8.0% to about 12.0% of caprylic/capric triglycerides;
(4) from about 4.0% to about 6.0% of PEG-20 almond glyceride;
(5) from about 4.0% to about 6.0% of polyethylene glycol;
(6) from about 40% to about 60% of sodium polyacrylate/polyacrylic acid;
(7) from about 0.024% to about 0.036% of lactoperoxidase;
(8) from about 0.0040% to about 0.0060% of myeloperoxidase;
(9) from about 0.024% to about 0.036% of glucose oxidase;
(10) from about 0.012% to about 0.018% of lactoferrin;
(11) from about 0.012% to about 0.018% of lysozyme; and
(12) from about 0.004% to about 0.006% of potassium thiocyanate.
   Preferably, Formulation 7 comprises:
(1) about 20% of 99% glycerol;
(2) about 10.0% of tripropylene glycol;
(3) about 10.0% of caprylic/capric triglycerides;
(4) about 5.0% of PEG-20 almond glyceride;
(5) about 5.0% of polyethylene glycol;
(6) about 50% of sodium polyacrylate/polyacrylic acid;
(7) about 0.030% of lactoperoxidase;
(8) about 0.0050% of myeloperoxidase;
(9) about 0.030% of glucose oxidase;
(10) about 0.015% of lactoferrin;
(11) about 0.015% of lysozyme; and
(12) about 0.005% of potassium thiocyanate.

Formulation 8 is a non-aqueous enzymatic lubricant in thick gel form.

Typically, Formulation 8 comprises:
(1) from about 24.8% to about 37.2% of PEG-40 stearate;
(2) from about 26.4% to about 39.6% of 99% glycerol;
(3) from about 5.6% to about 8.4% of tripropylene glycol;
(4) from about 2.88% to about 4.32% of caprylic/capric triglycerides;
(5) from about 5.68% to about 8.52% of PEG-20 almond glyceride;
(6) from about 5.68% to about 8.52% of polyethylene glycol;
(7) from about 8.88% to about 13.32% of sodium polyacrylate/polyacrylic acid;
(8) from about 0.024% to about 0.036% of aloe vera;
(9) from about 0.028% to about 0.042% of lactoperoxidase;
(10) from about 0.024% to about 0.036% of glucose oxidase;
(11) from about 0.012% to about 0.018% of lactoferrin;
(12) from about 0.012% to about 0.018% of lysozyme; and
(13) from about 0.004% to about 0.006% of potassium thiocyanate.
   Preferably, Formulation 8 comprises:
(1) about 31% of PEG-40 stearate;
(2) about 33% of 99% glycerol;
(3) about 7.0% of tripropylene glycol;
(4) about 3.6% of caprylic/capric triglycerides;
(5) about 7.1% of PEG-20 almond glyceride;
(6) about 7.1% of polyethylene glycol;
(7) about 11.1% of sodium polyacrylate/polyacrylic acid;
(8) about 0.030% of aloe vera;
(9) about 0.035% of lactoperoxidase;
(10) about 0.030% of glucose oxidase;
(11) about 0.015% of lactoferrin;
(12) about 0.015% of lysozyme; and
(13) about 0.005% of potassium thiocyanate.

Formulation 9 is a non-aqueous enzymatic lubricant in thick gel form.

Typically, Formulation 9 comprises:
(1) from about 16% to about 24% of beeswax;
(2) from about 27.2% to about 40.8% of 99% glycerol;
(3) from about 5.6% to about 8.4% of tripropylene glycol;
(4) from about 10.88% to about 16.32% of caprylic/capric triglycerides;
(5) from about 5.68% to about 8.52% of PEG-20 almond glyceride;
(6) from about 5.68% to about 8.52% of polyethylene glycol;
(7) from about 8.88% to about 13.32% of sodium polyacrylate/polyacrylic acid;
(8) from about 0.028% to about 0.042% of lactoperoxidase;
(9) from about 0.024% to about 0.036% of glucose oxidase;
(10) from about 0.012% to about 0.018% of lactoferrin;
(11) from about 0.012% to about 0.018% of lysozyme; and
(12) from about 0.004% to about 0.006% of potassium thiocyanate.
   Preferably, Formulation 9 comprises:
(1) about 20% of beeswax;
(2) about 34% of 99% glycerol;
(3) about 7.0% of tripropylene glycol;
(4) about 13.6% of caprylic/capric triglycerides;
(5) about 7.1% of PEG-20 almond glyceride;
(6) about 7.1% of polyethylene glycol;
(7) about 11.1% of sodium polyacrylate/polyacrylic acid;
(8) about 0.035% of lactoperoxidase;
(9) about 0.030% of glucose oxidase;
(10) about 0.015% of lactoferrin;
(11) about 0.015% of lysozyme; and
(12) about 0.005% of potassium thiocyanate.

Formulation 10 is a non-aqueous enzymatic lubricant in thick gel form.

Typically, Formulation 10 comprises:
(1) from about 24.8% to about 37.2% of PEG-40 stearate;
(2) from about 26.4% to about 39.6% of 99% glycerol;
(3) from about 5.6% to about 8.4% of tripropylene glycol;
(4) from about 2.88% to about 4.32% of caprylic/capric triglycerides;
(5) from about 5.68 to about 8.52% of PEG-20 almond glyceride;
(6) from about 5.68% to about 8.52% of polyethylene glycol;
(7) from about 8.88% to about 13.32% of sodium polyacrylate/polyacrylic acid;
(8) from about 0.024% to about 0.036% of aloe vera;
(9) from about 0.008% to about 0.012% of sodium phosphate;
(10) from about 0.028% to about 0.042% of lactoperoxidase;
(11) from about 0.024% to about 0.036% of glucose oxidase;
(12) from about 0.012% to about 0.018% of lactoferrin;
(13) from about 0.012% to about 0.018% of lysozyme; and
(14) from about 0.004% to about 0.006% of potassium thiocyanate.
   Preferably, Formulation 10 comprises:
(1) about 31% of PEG-40 stearate;
(2) about 33% of 99% glycerol;
(3) about 7.0% of tripropylene glycol;
(4) about 3.6% of caprylic/capric triglycerides;
(5) about 7.1% of PEG-20 almond glyceride;
(6) about 7.1% of polyethylene glycol;
(7) about 11.1% of sodium polyacrylate/polyacrylic acid;
(8) about 0.030% of aloe vera;
(9) about 0.010% of sodium phosphate;
(10) about 0.035% of lactoperoxidase;
(11) about 0.030% of glucose oxidase;
(12) about 0.015% of lactoferrin;
(13) about 0.015% of lysozyme; and
(14) about 0.005% of potassium thiocyanate.

Formulation 11 comprises a non-aqueous enzymatic lubricant in solid (suppository) form.

Typically, Formulation 11 comprises:
(1) from about 32% to about 48% of PEG-40 stearate;
(2) from about 18.4% to about 27.6% of 99% glycerol;
(3) from about 5.6% to about 8.4% of tripropylene glycol;
(4) from about 2.88% to about 4.32% of caprylic/capric triglycerides;
(5) from about 5.68% to about 8.52% of PEG-20 almond glyceride;
(6) from about 5.68% to about 8.52% of polyethylene glycol;
(7) from about 8.88% to about 13.32% of sodium polyacrylate/polyacrylic acid;
(8) from about 0.028% to about 0.042% of lactoperoxidase;
(9) from about 0.024% to about 0.036% of glucose oxidase;
(10) from about 0.012% to about 0.018% of lactoferrin;
(11) from about 0.012% to about 0.018% of lysozyme; and
(12) from about 0.004% to about 0.006% of potassium thiocyanate.
   Preferably, Formulation 11 comprises:
(1) about 40% of PEG-40 stearate;
(2) about 23% of 99% glycerol;
(3) about 7.0% of tripropylene glycol;
(4) about 3.6% of caprylic/capric triglycerides;
(5) about 7.1% of PEG-20 almond glyceride;
(6) about 7.1% of polyethylene glycol;
(7) about 11.1% of sodium polyacrylate/polyacryic acid;
(8) about 0.035% of lactoperoxidase;
(9) about 0.030% of glucose oxidase;
(10) about 0.015% of lactoferrin;
(11) about 0.015% of lysozyme; and
(12) about 0.005% of potassium thiocyanate.

Formulation 12 comprises a non-aqueous enzymatic lubricant in solid (suppository) form.

Typically, Formulation 12 comprises:
(1) from about 26.16% to about 39.24% of PEG-40 stearate;
(2) from about 22.0% to about 33.0% of 99% glycerol;
(3) from about 9.04% to about 13.56% of tripropylene glycol;
(4) from about 4.48% to about 6.72% of caprylic/capric triglycerides;
(5) from about 9.12% to about 13.68% of PEG-20 almond glyceride;
(6) from about 9.04% to about 13.56% of polyethylene glycol;
(7) from about 0.028% to about 0.042% of lactoperoxidase;
(8) from about 0.024% to about 0.036% of glucose oxidase;
(9) from about 0.012% to about 0.018% of lactoferrin; and
(10) from about 0.012% to about 0.018% of lysozyme.
   Preferably, Formulation 12 comprises:
(1) about 32.7% of PEG-40 stearate;
(2) about 27.5% of 99% glycerol;
(3) about 11.3% of tripropylene glycol;
(4) about 5.6% of caprylic/capric triglycerides;
(5) about 11.4% of PEG-20 almond glyceride;
(6) about 11.3% of polyethylene glycol;
(7) about 0.035% of lactoperoxidase;
(8) about 0.030% of glucose oxidase;
(9) about 0.015% of lactoferrin; and
(10) about 0.015% of lysozyme.

Formulation 13 is a non-aqueous enzymatic lubricant in thick gel form.

Typically, Formulation 13 comprises:
(1) from about 20% to about 30% of Polawax;
(2) from about 24% to about 36% of 99% glycerol;
(3) from about 20% to about 30% of tripropylene glycol;
(4) from about 8.0% to about 12.0% of caprylic/capric triglycerides;
(5) from about 8.0% to about 12.0% of PEG-20 almond glyceride;
(6) from about 0.028% to about 0.042% of lactoperoxidase; and
(7) from about 0.024% to about 0.036% of glucose oxidase.
   Preferably, Formulation 13 comprises:
(1) about 25% of Polawax;
(2) about 30% of 99% glycerol;
(3) about 25% of tripropylene glycol;
(4) about 10% of caprylic/capric triglycerides;
(5) about 10% of PEG-20 almond glyceride;
(6) about 0.035% of lactoperoxidase; and
(7) about 0.030% of glucose oxidase.

Formulation 14 is a non-aqueous enzymatic lubricant in thick gel form.

Typically, Formulation 14 comprises:
(1) from about 16% to about 24% of Polawax;
(2) from about 8.88% to about 13.32% of sodium polyacrylate/polyacrylic acid;
(3) from about 16% to about 24% of 99% glycerol;
(4) from about 20% to about 30% of tripropylene glycol;
(5) from about 8.0% to about 12.0% of caprylic/capric triglycerides;
(6) from about 8.0% to about 12.0% of PEG-20 almond glyceride;
(7) from about 0.028% to about 0.042% of lactoperoxidase;
(8) from about 0.024% to about 0.036% of glucose oxidase;
(9) from about 0.0128% to about 0.0192% of lactoferrin; and
(10) from about 0.0128% to about 0.0192% of lysozyme.
   Preferably, Formulation 14 comprises:
(1) about 20% of Polawax;
(2) about 11.1% of sodium polyacrylate/polyacrylic acid;
(3) about 20% of 99% glycerol;
(4) about 25% of tripropylene glycol;
(5) about 10.0% of caprylic/capric triglycerides;
(6) about 10.0% of PEG-20 almond glyceride;
(7) about 0.035% of lactoperoxidase;
(8) about 0.030% of glucose oxidase;
(9) about 0.016% of lactoferrin; and
(10) about 0.016% of lysozyme.

Formulation 15 is a non-aqueous enzymatic lubricant in gel form.

Typically, Formulation 15 comprises:
(1) from about 8.0% to about 12.0% of propylene glycol;
(2) from about 16% to about 24% of tripropylene glycol;
(3) from about 4.0% to about 6.0% of PEG-20 almond glyceride;
(4) from about 4.0% to about 6.0% of polyethylene glycol;
(5) from about 40% to about 60% of sodium polyacrylate/polyacrylic acid;
(6) from about 0.024% to about 0.036% of lactoperoxidase;
(7) from about 0.004% to about 0.006% of myeloperoxidase;
(8) from about 0.024% to about 0.038% of glucose oxidase;
(9) from about 0.012% to about 0.018% of lactoferrin;
(10) from about 0.012% to about 0.018% of lysozyme; and
(11) from about 0.004% to about 0.006% of potassium thiocyanate.
   Preferably, Formulation 15 comprises:
(1) about 10.0% of propylene glycol;
(2) about 20% of tripropylene glycol;
(3) about 5.0% of PEG-20 almond glyceride;
(4) about 5.0% of polyethylene glycol;
(5) about 50% of sodium polyacrylate/polyacrylic acid;
(6) about 0.030% of lactoperoxidase;
(7) about 0.005% of myeloperoxidase;
(8) about 0.030% of glucose oxidase;
(9) about 0.015% of lactoferrin;
(10) about 0.015% of lysozyme; and
(11) about 0.005% of potassium thiocyanate.

Formulation 16 is a non-aqueous enzymatic lubricant in solid (suppository) form.

Typically, Formulation 16 comprises:
(1) from about 32% to about 48% of PEG-40 stearate;
(2) from about 8.8% to about 13.2% of dipropylene glycol;
(3) from about 15.2% to about 22.8% of tripropylene glycol;
(4) from about 2.88% to about 4.32% of caprylic/capric triglycerides;
(5) from about 5.68% to about 8.52% of PEG-20 almond glyceride;
(6) from about 5.68% to about 8.52% of polyethylene glycol;
(7) from about 8.8% to about 13.2% of sodium polyacrylate/polyacrylic acid;
(8) from about 0.028% to about 0.042% of lactoperoxidase;
(9) from about 0.024% to about 0.036% of glucose oxidase;
(10) from about 0.012% to about 0.018% of lactoferrin;
(11) from about 0.012% to about 0.018% of lysozyme; and
(12) from about 0.004% to about 0.006% of potassium thiocyanate.
   Preferably, Formulation 16 comprises:
(1) about 40% of PEG 40 stearate;
(2) about 11.1% of dipropylene glycol;
(3) about 19% of tripropylene glycol;
(4) about 3.6% of caprylic/capric triglycerides;
(5) about 7.1% of PEG-20 almond glyceride;
(6) about 7.1% of polyethylene glycol;
(7) about 11.1% of sodium polyacrylate/polyacrylic acid;
(8) about 0.035% of lactoperoxidase;
(9) about 0.030% of glucose oxidase;
(10) about 0.015% of lactoferrin;
(11) about 0.015% of lysozyme; and
(12) about 0.005% of potassium thiocyanate.

Other formulations can be prepared that are similar to the ones described in detail above.

Formulations according to the present invention are effective in treating vaginal diseases, particularly those of bacterial and fungal etiology. They act by enzymatic activity. They do not cause side effects and do not interfere with other treatments, such as antibacterial and antifungal agents. Their enzymatic activity enhances the vagina's natural defenses.

Formulations according to the present invention have industrial applicability because of their use for treating vaginal diseases or for the preparation of a medicament for the treatment of vaginal diseases.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of ordinary skill in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. Use of a composition formulated for vaginal administration to treat a vaginal condition, the composition comprising:
(a) a peroxidase enzyme selected from the group consisting of lactoperoxidase, horseradish peroxidase, myeloperoxidase, eosinophil peroxidase and glutathione peroxidase;
(b) a salt that is an alkali metal salt of an anion selected from the group consisting of thiocyanate, iodate and chlorate;
(c) at least one of an ingredient selected from the group consisting of lysozyme, lactoferrin and a steroid; and
(d) an amount less than 15% of PEG 20,
wherein the composition is a lubricant, further wherein the lubricant has a pH suitable for maintaining a stable Lactobacillus population within a vagina.

2. The use of claim 1, wherein the composition further comprises lactic acid.

3. The use of claim 1, wherein the composition further comprises an oxidoreductase enzyme selected from the group consisting of glucose oxidase, galactose oxidase, urate oxidase, choline oxidase, D-amino acid oxidase, D-glutamate oxidase, glycine oxidase, glycolic oxidase, L-sorbose oxidase, alcohol oxidase, and amine oxidase.

4. The use of claim 1, wherein the composition further comprises an effective amount of an inhibitor that is specific for catalase.

5. The composition of claim 4, wherein the inhibitor that is specific for catalase is a salt of ascorbic acid.

6. The use of claim 1, wherein the composition further comprises a quantity of an aminohexose effective in increasing the yield or accumulation of biocide formed.

7. The use of claim 1, wherein the composition further comprises a buffering agent.

8. The composition of claim 7, wherein the buffering agent is selected from the group consisting of sodium stearate, potassium stearate, and calcium stearate.

9. A composition formulated for vaginal administration, comprising:
(a) a peroxidase enzyme selected from the group consisting of lactoperoxidase, horseradish peroxidase, myeloperoxidase, eosinophil peroxidase and glutathione peroxidase;
(b) a salt that is an alkali metal salt of an anion selected from the group consisting of thiocyanate, iodate and chlorate;
(c) at least one of an ingredient selected from the group consisting of lysozyme, lactoferrin and a steroid;
(d) an amount less than 15% of PEG-20; and
(e) an aqueous medium in which the peroxidase enzyme and the salt are stable, such that the composition is suitable for vaginal administration and further wherein the composition is a media wherein the media is a lubricant and has a pH suitable for maintaining a stable Lactobacillus population within a vagina.
